(19) United States
(12) Patent Application Publication
(10) Pub. No.: US 2025/0111317 A1
(43) Pub. Date: Apr. 3, 2025

(54) System and Method for Measuring and Managing Health Risks in an Enclosed Space

(71) Applicant: HEALTHY SPACE HOLDINGS, West Molesey Surrey (GB)

(72) Inventors: Ralph Izod, London (GB); Steve Burch, London (GB)

(21) Appl. No.: 18/730,386

(22) PCT Filed: Jan. 23, 2023
(86) PCT No.: PCT/EP2023/051591

§ 371 Date: Jul. 19, 2024

(30) Foreign Application Priority Data
Jan. 21, 2022 (GB) 2200814.8

(51) Int. Cl. G06Q 10/0635; G05B 15/02; G16H 50/80

(52) U.S. Cl. CPC G06Q 10/0635; G05B 15/02; G16H 50/80; G05B 2219/2614; G05B 2219/2642

(57) ABSTRACT

A system for mitigating risk in an enclosed space comprises: a processor unit having a rules engine and a workflow engine; an infrastructure library accessible by the processor unit and holding a plurality of available interventions which can be enacted by the system; a plurality of environmental sensors, each arranged to supply the processor unit with sensor signals indicating a sensed risk factor of the enclosed space; a workflow interface arranged to instruct the operation of a workflow which enacts an intervention; wherein the rules engine is configured to process the sensor signals from the plurality of sensors against a set of rules to determine if the risk is elevated to a level where mitigating action is required; wherein the workflow engine is configured to select one or more of the available interventions from the infrastructure library when intervention is required; wherein the processor unit is configured to generate a workflow instruction signal when a workflow has been selected and to send that workflow instruction signal to the workflow interface;

The present invention relates to a system, and a computer-implemented method, of measuring specified health risks to people within a building or other enclosed environment, leading to operation of mitigating actions.

It is well known to place sensors within a building environment to measure temperature, and to respond to the measured temperature being too hot or too cold by increasing cooling or heating to that environment with the aim being to keep the temperature within a target temperature band. Other ambient conditions, such carbon monoxide can also be measured, and action taken to avoid the environment from becoming hazardous.

In addition, with the spread of Covid-19, it has started to become common to protect against viruses and other microbiological hazards by regularly swabbing surfaces, and if they are detected, taking action to reduce the risk from them by increasing cleaning However, these known systems are very basic, and are limited in their effectiveness.

Enclosed spaces, such as buildings, trains, buses and the like pose a variety of health and environmental risks to the health of people occupying that space. Risk factors include:

Elevated levels of organic compounds residing on surfaces, which are an indication of elevated levels of pathogens (e.g. viruses and bacteria which might cause disease) on surfaces and in the air, thereby representing an elevated level of risk of transmission of pathogens to people from contact with surfaces and from exposure to the air;
Elevated levels of carbon dioxide in the air, which provides an indication of an elevated risk of transmission of pathogens to people from the air;
Elevated or low levels of humidity in the air, which research shows provides an indication of an elevated health risk, for example transmission of pathogens to people from the air;
Elevated levels of carbon dioxide in the air, which impairs cognitive function, learning, productivity and well-being;
Elevated levels of particulate matter, including particulate matter PM2.5 and particulate matter PM10 which can irritate lungs, agitate pre-existing conditions, cause heart attacks and cancer and, based on latest research, might be linked to dementia;
Elevated levels of airborne chemicals including Volatile Organic Compounds (VOCs) which can cause long-term chronic health effects;
Elevated levels of density and proximity of people within the enclosed space;
Movement (flow and dwell time) of people in defined spaces, which indicates an elevated risk of transmission of pathogens between people;
Elevated levels of radon in the air—research shows that radon produces a radioactive dust in the air we breathe which becomes trapped in the lungs causing damage and increasing the risk of lung cancer;
Elevated or low air temperature—this can impact the well-being and comfort of people within enclosed spaces; and

Elevated or low light levels—this may impact on the risks of viral transmission as well as levels of well-being and comfort of people within an enclosed space.

The present invention intends to reduce the health risks that people are exposed to.

According to a first aspect of the invention, a system for mitigating risk in an enclosed space comprises: a processor unit having a rules engine and a workflow engine; an infrastructure library accessible by the processor unit and holding a plurality of available interventions which can be enacted by the system to mitigate risk; a plurality of environmental sensors, each arranged to supply the processor unit with sensor signals indicating a sensed risk factor of the enclosed space; and a workflow interface arranged to instruct the operation of a workflow which enacts an intervention; wherein the rules engine is configured to process the sensor signals from the plurality of sensors against a set of rules to determine if the risk is elevated to an elevated risk level where mitigating action is required; wherein the workflow engine 20 is configured to select one or more of the available interventions from the infrastructure library when the risk is elevated to the elevated risk level where mitigating action is required; and wherein the processor unit is configured to generate a workflow instruction signal when an intervention has been selected and to send that workflow instruction signal to the workflow interface.

Preferably, the rules engine is arranged to generate a technical healthiness rating based on its determination of risk, the technical healthiness rating being an objective technical measure of the risk factors in the enclosed space. There might also be an indicator for generating an indication of the technical healthiness rating.

The system advantageously includes a standards library which holds a set of pre-programmed standards for the enclosed space to meet. It is also advantageous that the system includes a library of modifiers including at least one of: risk modifiers; rating modifiers, workflow modifiers; and energy efficiency modifiers.

The rules engine may be configured to modify the elevated risk level of the set of rules based on the risk modifier in order to modify the risk level at which mitigation action is required. The workflow engine may be configured to modify the intervention based on the workflow modifiers and/or energy efficiency modifiers in order to modify the intervention that is required.

It is also advantageous for the system to include means for sourcing external environmental information and supplying that information to the processor unit. For example, levels of humidity, particulate matter and other pollutants such as Ozone, sulphur dioxide or nitrogen oxides in the external air may increase health risks in enclosed spaces as outdoor passes into those enclosed spaces.

The system may further comprise a sensor data storage for storing sensor data corresponding to the sensor signals from the sensors over a period of time. Preferably, the sensor data storage can also be arranged to store the external environmental information over a period of time, although this could be stored in separate storage.

The workflow interface may be arranged to source verification that an intervention has been implemented.

The processor unit is preferably configured to analyse the effect of the intervention on the sensed risk factors of the enclosed space from the environmental sensors, and if further intervention is required, to select a further intervention.

A system preferably comprises a workflow response library which stores historical interventions which have taken place, together with the (i) verification and/or (ii) validation of those interventions. In this case, the processor can be further configured to analyse the effect of an intervention on the sensed risk factors of the space from the environmental sensors, and to record the result in the workflow response library; and the processor unit can be configured to use the contents of the sensor data storage (including external environmental information) and workflow response library when selecting one or more mitigation actions. The processor unit may include a machine learning or AI engine arranged to apply machine learning or AI to the analysis of the effect of an intervention on the sensed risk factors of the space. The processor unit or machine learning or AI engine are advantageously arranged to edit the set of rules in the rules engine and/or the workflow engine to change their responses. Advantageously, the editing of the set of rules in the rules engine and/or the workflow engine changes the risk modifiers, workflow modifiers, rating modifiers and/or energy efficiency modifiers.

The machine learning or AI engine can be arranged to analyse the sensor data, and advantageously, external environmental information as well, stored in the sensor data storage and to identify repeat instances where elevated risk is detected so as to cause the system to pre-empt the elevation of risks with the implementation of an intervention.

Preferably, the workflow interface is connected to a plurality of different systems for carrying out different interventions. These systems may include, but are not limited to, Building Management Systems (BMS), Heating Ventilation and Air Conditioning (HVAC) systems and other air handling systems, air purification or air treatment systems and/or devices that eradicate pathogens from the air, such as UV photocatalytic decontamination units, energy efficiency systems and processes, meeting room booking systems and desk occupancy systems.

According to a second aspect of the invention, a method of mitigating risk in an enclosed space comprises: processing, using a rules engine, the sensor signals from a plurality of environmental sensors, each sensor arranged to supply the rules engine with sensor signals indicating a sensed risk factor of the enclosed space against a set of rules to determine if the risk is elevated to an elevated risk level where mitigating action is required; selecting one or more available interventions which can be enacted by the system from an infrastructure library when the risk level is elevated to the elevated risk level where intervention is required; and generating a workflow instruction signal when an intervention has been selected and sending that workflow instruction signal to a workflow interface which is arranged to instruct the operation of a workflow which enacts the selected intervention.

The invention will now be described in more detail with reference to embodiments which are described by way of example only, and with reference to the drawings in which:

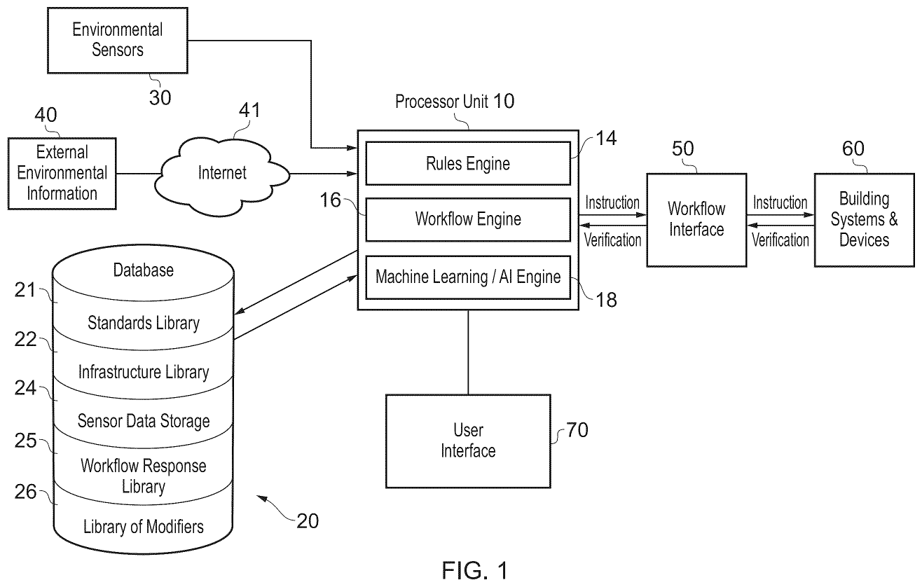

FIG. 1 is a diagram of the system according to an embodiment of the invention;

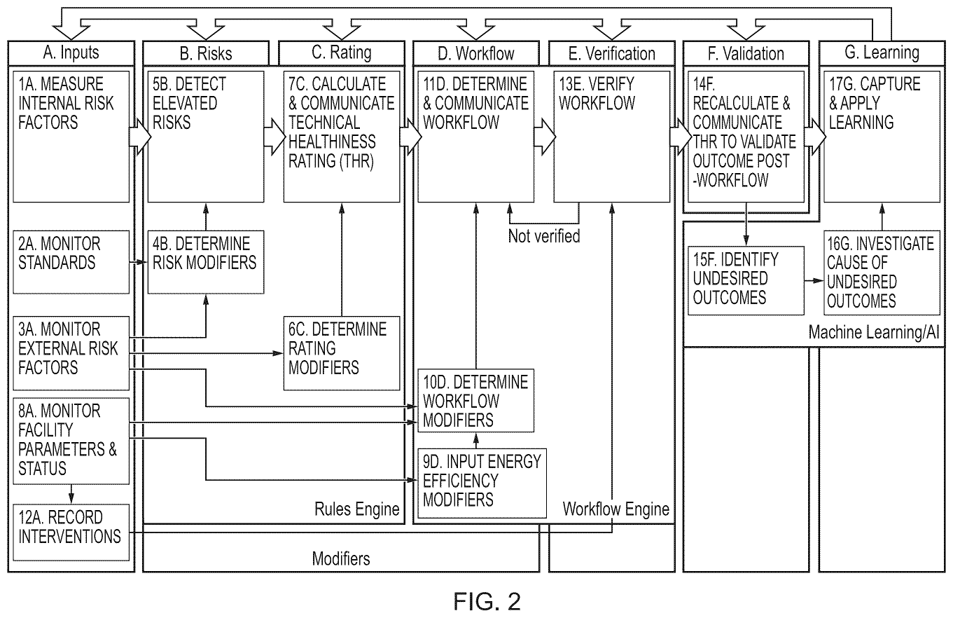

FIG. 2 is a chart of the methodology used in the present invention;

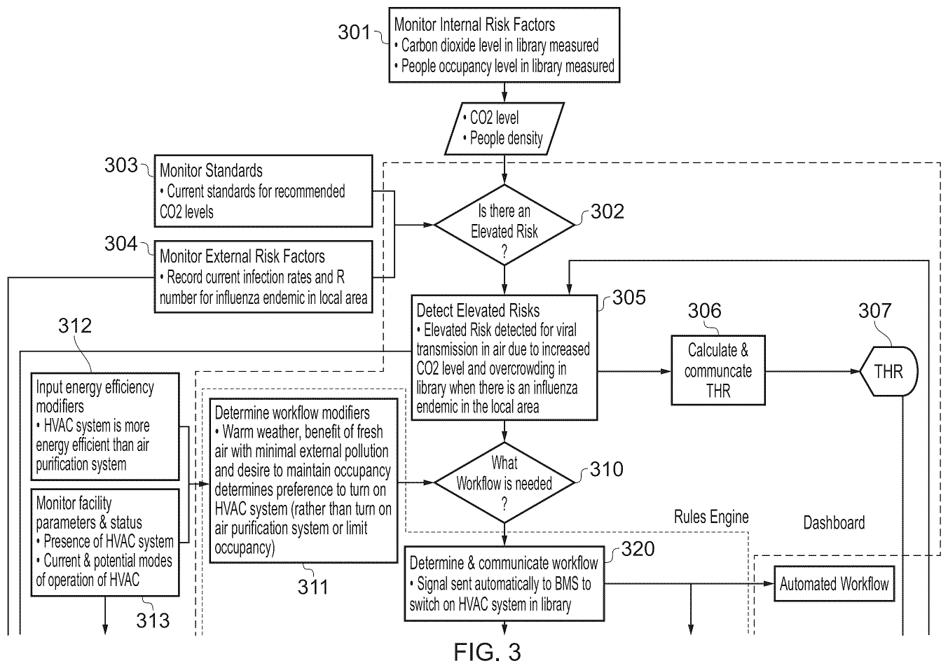

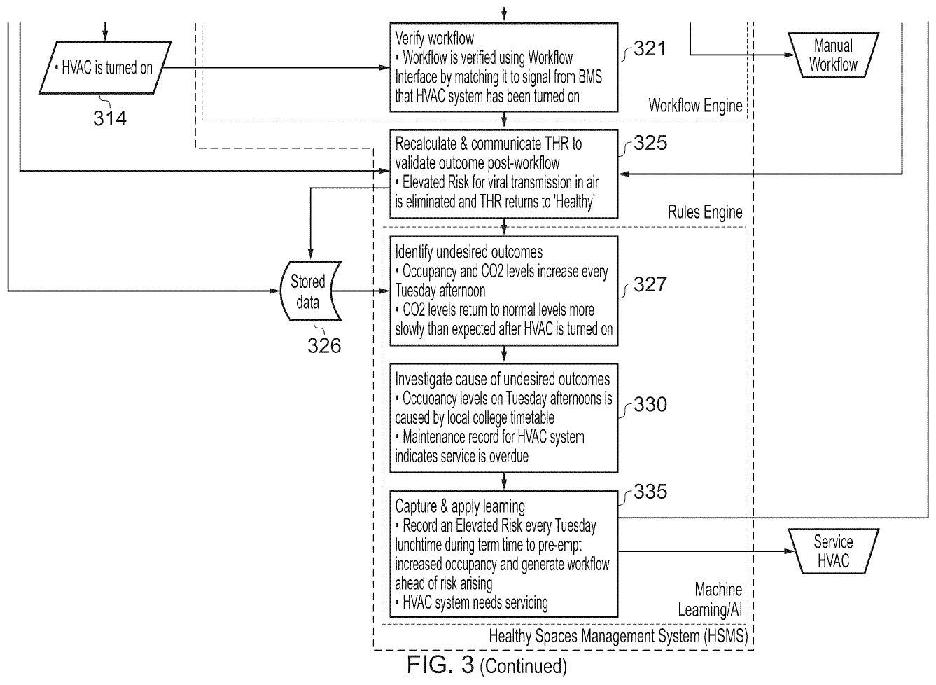

FIG. 3 is a flow diagram of the operation of the present application applied to a first example;

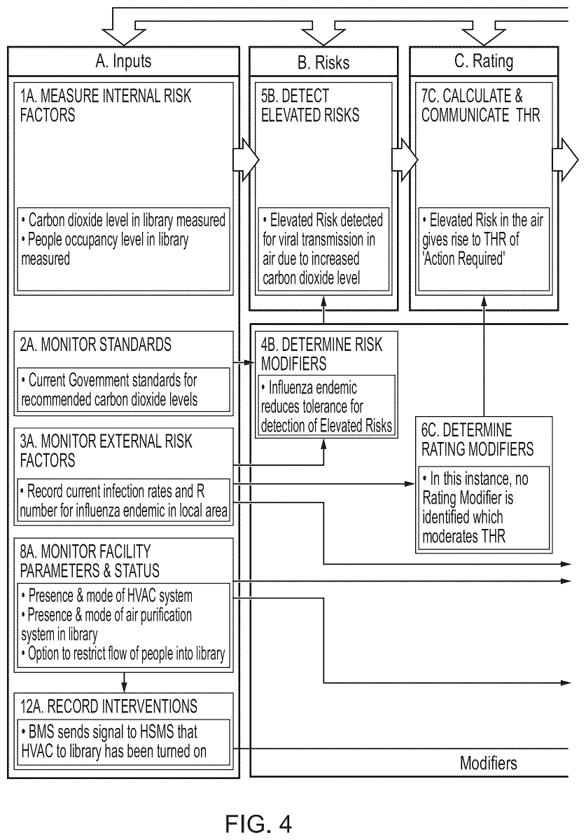

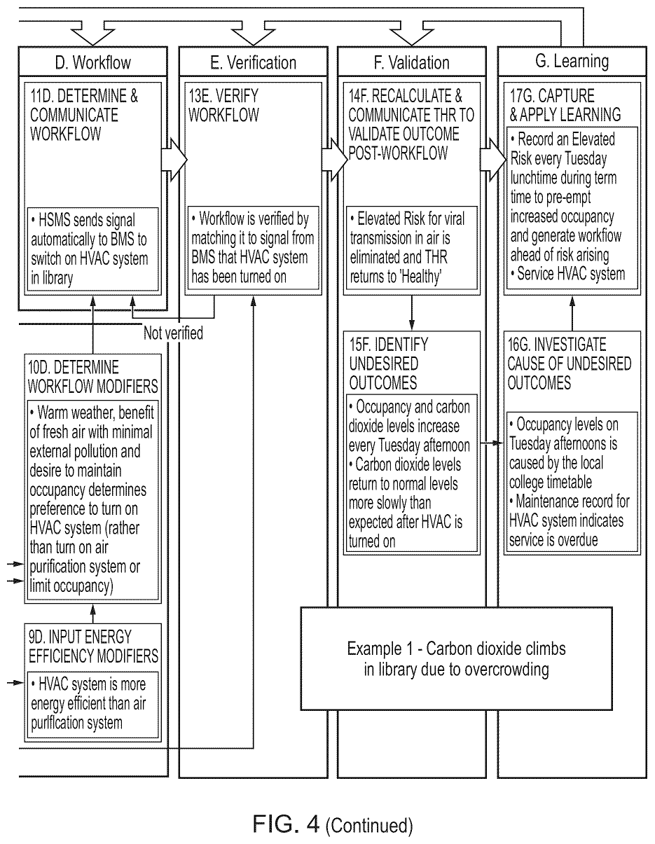

FIG. 4 is a chart of the methodology used in the first example;

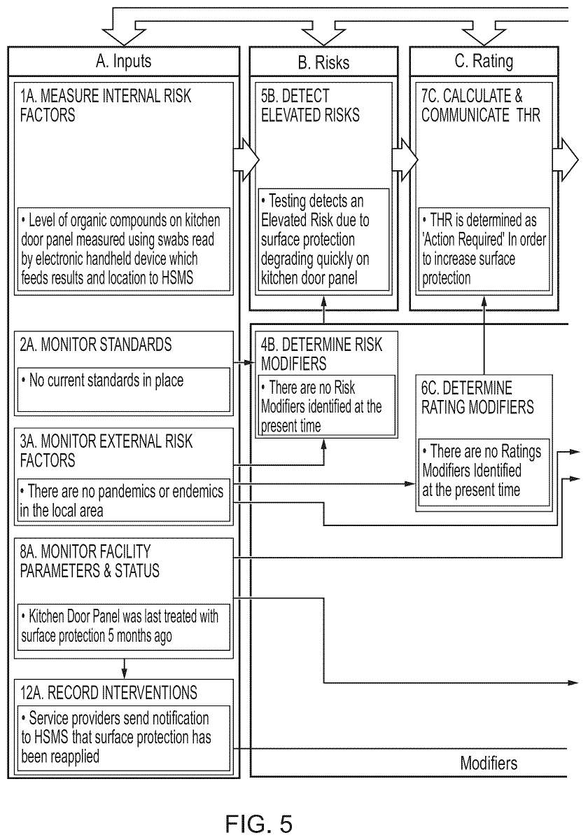

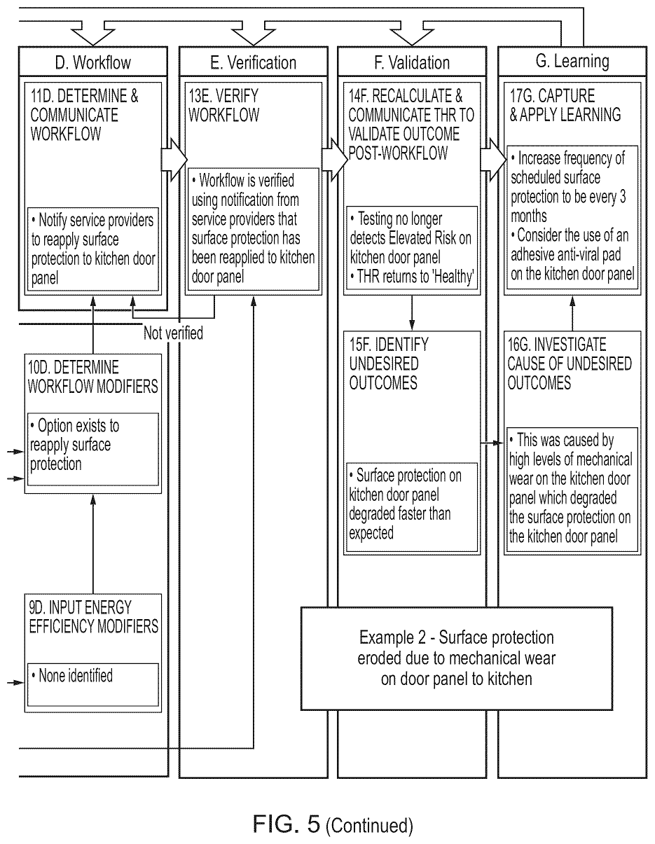

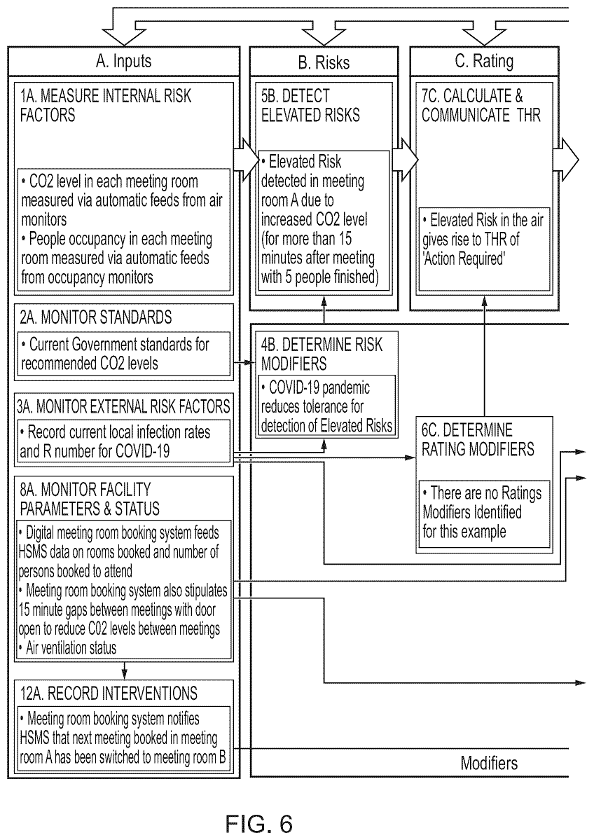

FIG. 5 is a chart of the methodology used in a second example;

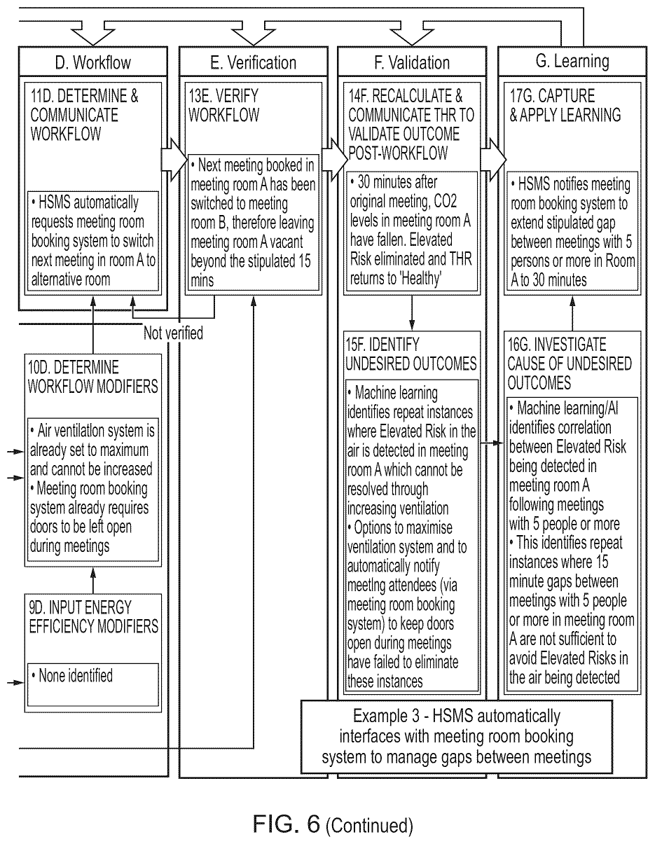

FIG. 6 is a chart of the methodology used in a third example;

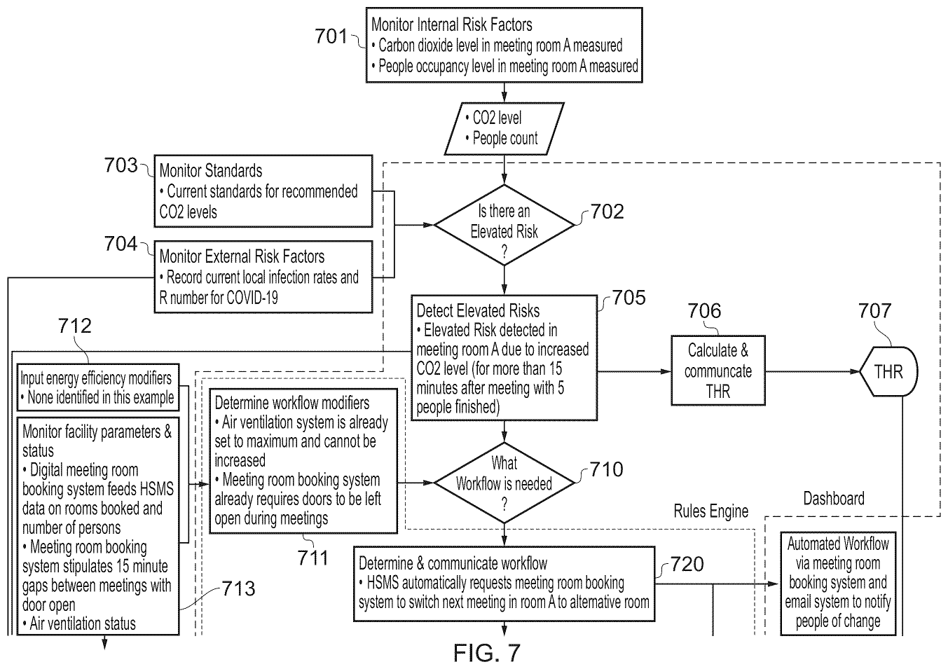

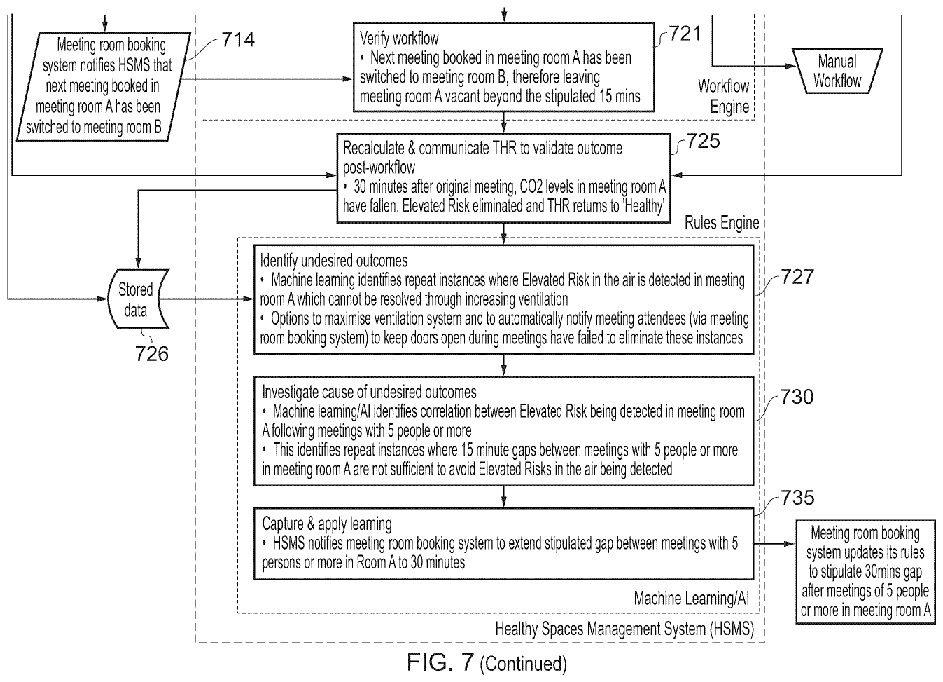

FIG. 7 is a flow diagram of the present invention applied to the third example;

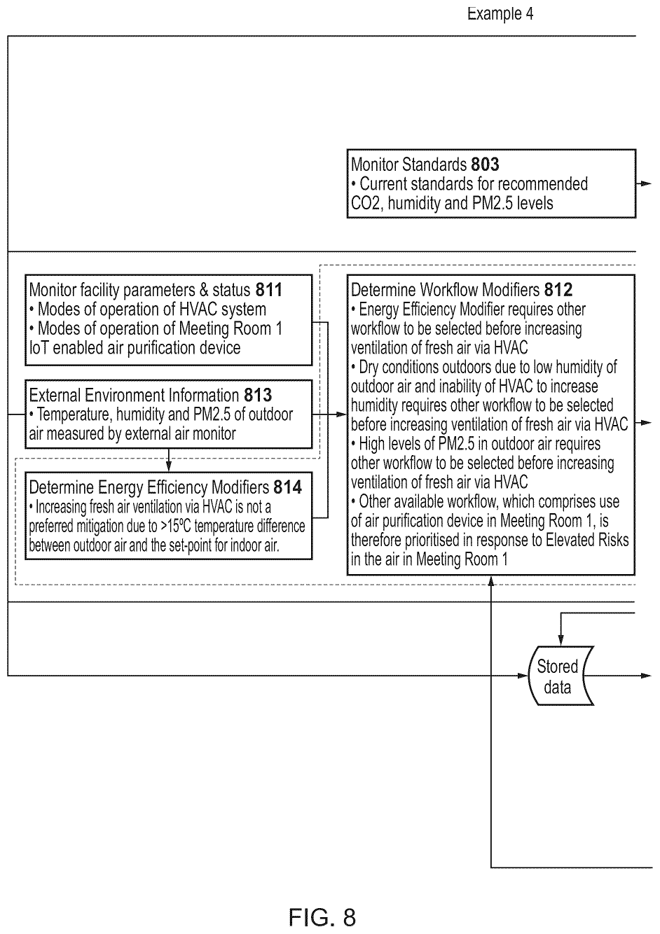

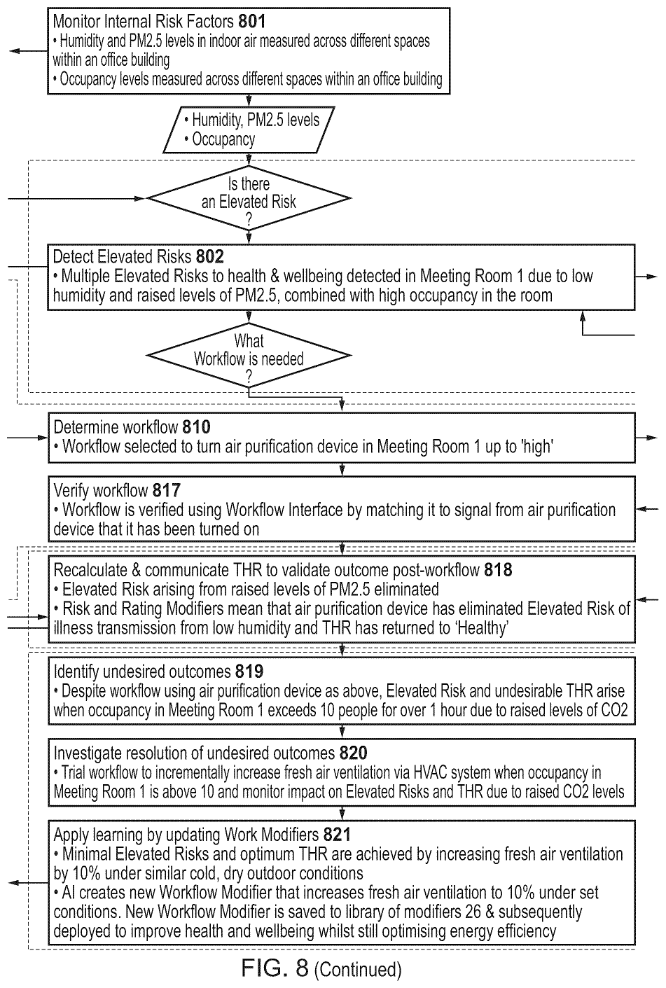

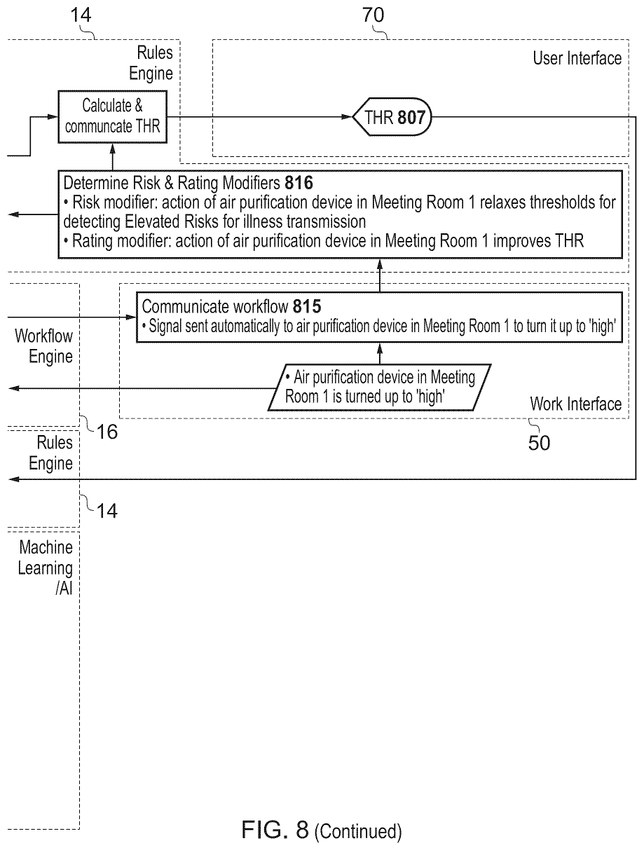

FIG. 8 is a flow diagram of a fourth example of the invention; and

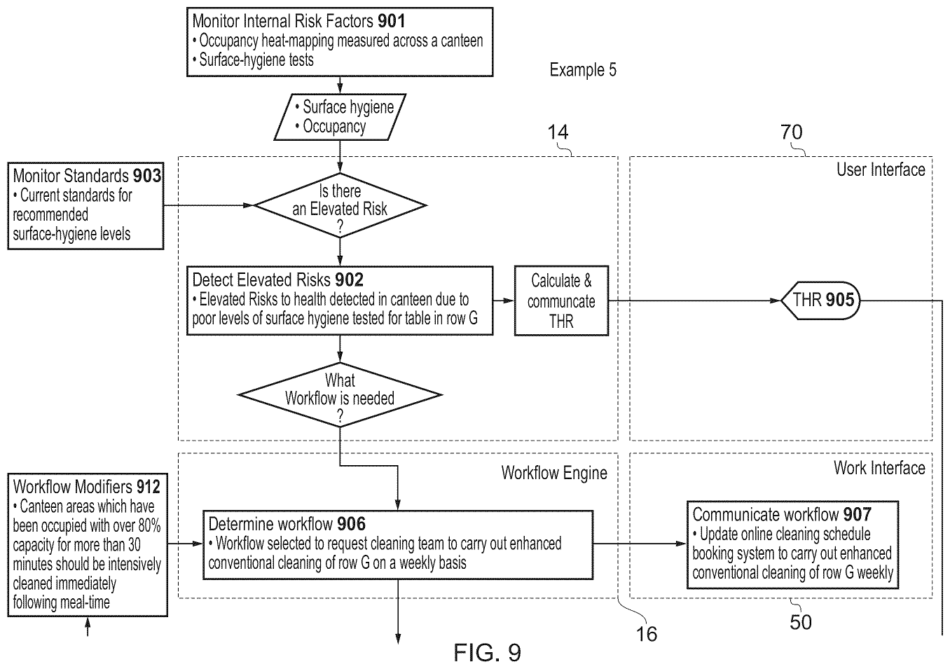

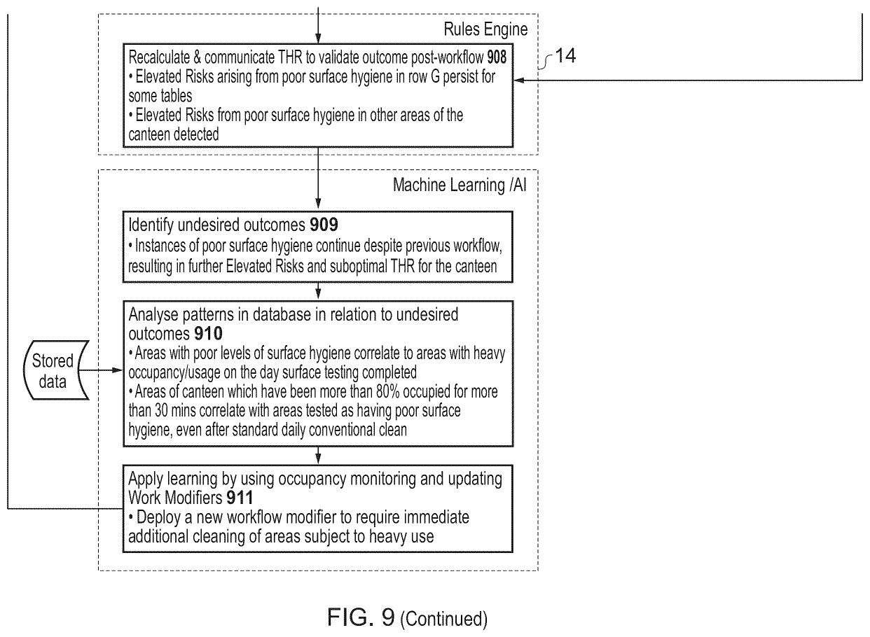

FIG. 9 is a flow diagram of a fifth example of the invention.

FIG. 1 shows a system for monitoring an enclosed space for health risks and for taking action to mitigate those risks. The system also provides a user interface for those responsible for managing the enclosed space to help them better manage the risks.

The system in FIG. 1 includes: a processor unit 10 having a rules engine 14 a workflow engine 16 and a machine learning/AI engine 18; a database 20 connected to the processor unit 10; a plurality of environmental sensors 30 connected to the processor unit 10; a source of external environmental information 40 connected to the processor unit 10 via the Internet 41 by which the processor unit 10 sources external environmental information; a workflow interface 50 connected to the processor unit 10; building systems and devices 60 connected to the workflow interface 50; and a user interface 70 connected to the processor unit.

The rules engine 14 of the processor unit 10 is able to determine the risk at any point in time by assessing the sensor signals from the environmental sensors 30, together with any risk modifiers (see below). The rules engine 14 can be arranged in the form of a map of the sensor signals from the environmental sensors and the risk modifiers and translates them into a rating which can involve a rating that the risk is elevated. The rules engine may be arranged so that it is editable (either manually or via machine learning and artificial intelligence). As will be discussed below, the rules engine 14 of the processor unit may be capable of assessing the effect of interventions so that, if an intervention proves not to be effective, then the rules can be changed to enable a different intervention to be selected in that situation in future.

The workflow engine 16 of the processor unit 10 is able to determine the intervention to be taken in the event that the rules engine 14 determines that there is an elevated risk in order to keep the enclosed space from exceeding the standards set out in the standards library 21.

The environmental sensors 30 are a set of sensors which operate within the enclosed space and which, across the different sensors, measure more than one risk factor. Examples of the sensors include: CO2 sensors for measuring the levels of CO2 within the air within the enclosed space; humidity sensors for measuring the humidity of air within the enclosed space; chemical sensors measuring for certain chemicals, such as VOCs within the air; particulate sensors for measuring airborne particulates of particular sizes, such as PM1.0, PM2.5 or PM10 particulates; sensors measuring occupancy and density of people within a space; people counting sensors; and surface swabbing sensors which are able to measure the presence of organic particles on the surface being tested. The environmental sensors generate sensor signals indicative of the measured risk factors and direct the sensor signals to the processor unit 10 either directly or via Application Programming Interface (API) or other electronic means over the Internet. The sensors could include sensors which are already in place for other purposes, for example, fire detectors, temperature sensors from HVAC systems, and cameras and gates from access control systems.

The External Environmental Information 40 may be provided by a variety of different sources over the internet, including external air monitors via API or subscription-based services providing data on weather, pollution and other aspect of outdoor air quality in local areas. For example, an air monitor may be installed on a roof of a building to measure VOCs and/or particulates near to the outdoor inlet of an HVAC system. The measured levels of VOCs and/or particulates may then be directed to the processor unit 10 via an API over the Internet.

The database 20 includes several libraries of data that can be accessed and used by the processor unit 10. The database includes a standards library 21 which stores a set of pre-programmed standards, including a set of standards which the enclosed space must meet. 25 Those standards could originate from governments or from international standards bodies, combined with the latest relevant industry standards and scientific research that relate to the health risks associated with enclosed spaces. Beyond any regulatory requirements, management of enclosed spaces may also set their own internal standards. These standards might also include external risk factors, by which we mean elements in the external environment 30 beyond the enclosed space that are measurable and may impact on the risks within the enclosed space. The standards can also include temporary restrictions related to pandemics. These standards can be used to define technical healthiness ratings against which the enclosed space can be assessed.

The database 20 also includes an infrastructure library 22 which contains extensive information concerning the enclosed space, including things such as: the floorplan of the enclosed space; the building layout; a digital model of the enclosed space, such as a BIM model or digital twin; information on the presence of a building management system or equivalent and its modes of operation; the presence, location and modes of operation of any filtration, ventilation or HVAC system, including the location of its inlets and outlets; the presence of any manual ventilation, including windows and doors that can be opened; the presence of any air purification or air treatment systems and/or machines that eradicate pathogens, such as UV photocatalytic decontamination units, from the air, including the modes of operation of such systems; energy efficiency systems and processes, including their modes of operation and their inputs, where they are not integrated into the building management system; room booking systems, including their modes of operation and their inputs; desk occupancy systems, including their modes of operation and their inputs,; maximum occupancy data for the enclosed space and for areas or rooms within that enclosed space; maximum permitted flow of people within the enclosed space; temporary internal requirements in place for people to take mitigating actions such as the wearing of masks and/or use of hand sanitiser in the facility; and the options for refilling of hand sanitiser stations in the facility. A list of all interventions that are available in that enclosed space is also stored in this database, based on the information listed above. The interventions will vary from enclosed space to enclosed space. Examples of interventions include: opening windows or doors to increase ventilation; each active mode of operation of any active air treatment system; each active mode of operation of any air filtration system; each active mode of operation of any HVAC system; each active mode of operation of any machine within the enclosed space that kills pathogens in the air; applying/re-applying surface protection in identified locations where an elevated risk is identified; mandating the wearing of masks; replenishing of hand sanitiser; reducing or restricting occupancy within the enclosed space, or a part of it (e.g. in meeting rooms via a meeting room booking system); restricting the movement of people within the enclosed space; and closing the whole enclosed space or a part of it. Each of these interventions is linked to a workflow action which the processor unit directs to the workflow interface 50 so that the workflow interface can effect the intervention.

The database includes sensor data storage 24 for storing historical sensor data 30 corresponding to the sensor signals from the sensors over a period of time, and for storing the historical external environmental data 40. The external environmental data could, of course, be stored in a separate store.

The database includes a workflow response library 25 which stores historical interventions which have taken place, together with the (i) verification and (ii) validation of those interventions. Validation is achieved by the measured effect of those interventions in terms of the sensor signals output by the environmental sensors. By collating, comparing, processing and analysing the data from the sensor data storage 24 and the workflow response library 25, machine learning and AI systems used on the data can improve the selection of the best intervention when an elevated risk is detected, and pre-empt elevated risks in the future before they arise. This will lead to improved technical healthiness ratings in future for the space.

The database 20 also includes a library of modifiers 26. There are four types of modifiers stored in the library of modifiers: risk modifiers; rating modifiers, workflow modifiers; and energy efficiency modifiers. The modifiers are determined by the processor unit 10, but are all stored in the library of modifiers 26, and the library is updated as they change. The modifiers are an important part of the invention because they are a very effective way to enable the system to adapt to changing conditions and multiple inputs, and because they can be used effectively in conjunction with AI and machine learning to cause more effective interventions to be enacted in any situation.

Firstly, the risk modifiers can be used to modify the level at which an elevated risk is determined and therefore intervention is needed, and the strength of an intervention. The type of intervention could also be modified. Circumstances might arise where it is appropriate to modify the level at which an elevated risk is detected or the resulting strength of intervention, for example, where there has been an outbreak of disease. The trigger for applying risk modifiers might include external environmental information sourced from outside the system. Examples of risk modifiers would be
Lower tolerance for increased levels of carbon dioxide and/or humidity in the air due to a change in the standards, for example, due to the presence of a pandemic or endemic in the local area where local authorities wish to apply more stringent restrictions or mitigations to reduce the spread of infection
Lower tolerance for increased levels of carbon dioxide and/or humidity in the air due to high levels of infection rates in the local population from a pandemic or endemic, and where management of facilities wish to apply more stringent mitigations to reduce the risk of infection
Lower tolerance for increased level levels of organic compounds detected on surfaces due to the presence of a pandemic or endemic in the local area, and where local authorities or management wish to apply more stringent mitigations to reduce the risk of infection
Lower tolerance for increased levels of organic compounds detected on surfaces due to high reported levels of infection rates in the local population from a pandemic or endemic
Lower tolerance for risk factors as a result of the demographics of a particular local area indicating greater susceptibility of the local population to pathogens, for example a high proportion of people post-retirement age in the local area
Lower tolerance for risk factors as a result of the demographics of those using a particular facility, for example a care home for the elderly or a ward for critically ill patients in a hospital
Lower or higher tolerance for risk factors as a result of changing patterns of use of a facility, for example a hospital operating theatre which is repurposed to space with a lower level of risk from infections.
Increased tolerance for risk factors as a result of air purification or air sterilisation systems being active in a particular space, for example higher levels of carbon dioxide may be acceptable in an office where air purification devices are operational

Real-time data on external risk factors and the external environmental information 40), is sourced by the system using automatic feeds and application programming interfaces (APIs) via the Internet 41. The external risk factors are things in the external environment beyond the enclosed space or premises containing that space, that are measurable and may impact on the health risks within the enclosed space. The following are some examples of the external environmental information:
Presence of pandemic or endemic
Local infection rates related to pandemic or endemic
Local levels of carbon dioxide
Local levels of PM10 and PM2.5
Local levels of pollen, ozone, sulphur dioxide and nitrous oxides in the air
External emissions of VOCs to the outdoor air in the local vicinity
The price of energy supplied to the premises
Local weather (e.g. ambient temperature and UV levels)
Season (e.g. month of year)
Humidity in local area.
Demographics of those using the facility in the local area and their susceptibility to pathogens

The processor unit 10 can use this information to automatically apply any appropriate risk modifiers to take account of external factors. For example, the presence of a pandemic 10 with a high infection rate in the local population will reduce the level of the risk factors that need to be measured before elevated risks are identified and interventions are applied by the processor unit 10.

Secondly, the processor unit 10 can use this information to determine rating modifiers, which are stored in the library of modifiers 26. Whilst the technical healthiness rating and the need for interventions is primarily driven by the measurement of the risk factors and the detection of elevated risks, there may be external risk factors in the external environment which may be beneficially used to modify the way in which the technical healthiness rating is categorised and reported. These external risk factors might be events in the external environment which increase risks factors within the enclosed space and/or lead to an elevated risk being detected, but where the risk factors within the enclosed space are still lower than those risks outside of the building. For example, the external environmental information 40 may show that there is significantly raised levels of PM2.5 outdoors in a city area which leads to a consequential rise in PM2.5s being detected indoors due to ventilation, but where the levels indoors remain healthier than those outdoors. In this example, the numerical technical healthiness rating will be reduced by the increase in PM2.5 indoors, however the way the technical healthiness rating is applied and presented can be modified to reflect the fact that it is still more healthy inside than outside. As an example the technical healthiness rating may be presented on an unmodified basis (i.e. using the levels of PM2.5 measured by the environmental sensors 30) and on a modified basis (i.e. then applying a rating modifier which reflects the relative level of PM2.5 indoors compared to the level outdoors). If the owners of the building were to invest in an air filtration system that removed PM2.5s from the air indoors, then the numerical technical healthiness rating would be improved to recognise the improvement in indoor air quality. Other high levels of external risk factors which might lead to applying rating modifiers are: increased humidity, high levels of other airborne particulate matter and VOCs. These are examples of where the risk factors within the enclosed space could be higher and lead to Elevated Risks being detected, but with the technical healthiness rating category reflecting the fact that users of the space are at less risk to their health by being indoors rather than outdoors.

Thirdly, the processor unit 10 can use this information to apply workflow modifiers, stored in the library of modifiers 26, which then influence the determination of the Workflow. Workflow modifiers are used in response to external environmental information and/or management preference which can influence the selection of the intervention, and thus the workflow that is implemented.

Workflow Modifiers are selected that are relevant to that space and the features of the building and related facilities it is contained within. For example, high levels of airborne particulate matter in the local area externally of the enclosed space may mean that a Workflow to increase ventilation using outdoor air is not an appropriate Intervention if this is resulting in high levels of particulate matter being detected indoors. In this example, the high levels of particulate matter outdoors would be detected using the external environmental information 40 so that the workflow engine 16 would use the library of modifiers 26 to apply a workflow modifier that selects workflow from the infrastructure library 22 which does not involve ventilation of outdoor air. In this case, an elevated risk from increased particulate matter indoors might result in the selection of workflow to increase the ventilation rates of internally recycled air (rather than fresh air) through the HVAC system, or the use of an in-room air purification device.

Fourthly, the energy efficiency modifiers are factors that influence the type of intervention that is determined in order to ensure that energy efficiency is taken into account. For example, it is important to ensure that an intervention which is more energy efficient and has the same level of efficacy in eliminating an elevated risk (and improving the technical healthiness rating) is prioritised over an intervention which is less energy efficient. Equally, the workflow engine 16 may apply an energy efficiency modifier to select an intervention that switches a system or device off or down when it is no longer needed and when that system or device is using energy. For example, where elevated levels of carbon dioxide in the air have fallen such that a previously elevated risk has been eliminated, the workflow engine 16 may use the external environmental information 40 which indicates cold (or warm) temperatures externally and apply an energy efficiency modifier to select an intervention that reduces the amount of cold (or hot) fresh air that is being ventilated into the premises through its HVAC system and must therefore be heated (or cooled). In this example, the workflow engine is therefore able to use the energy efficiency modifier to only select energy-intensive workflow when it is needed. The way in which this is achieved will vary widely depending on the type of building and its related facilities and uses. For example, building management systems increasingly consider energy efficiency. By integrating the present invention with the building management system in a building or facility, the building management system or other integrated software linked to the building management system can be used to provide automatic feeds which modify the type of intervention that is generated by the processor unit to reflect energy efficiency considerations. This ensures that an intervention is generated by the processor to keep spaces healthy whilst at the same time achieving energy efficiency objectives.

The user interface 70 provides an interface for those responsible for managing a defined space within a building or facility to help them better manage the risk. If a building management system or equivalent is present, this can be integrated as such an interface. The user interface 70 also provides information to people within the enclosed space, for example to indicate the technical healthiness rating determined by the processor unit. It will be understood that there is likely to be more than one user interface, the interfaces being arranged to be appropriate for the particular user. The user interface could be one or more of: a touchscreen located within the enclosed space; a computer application; a mobile phone or tablet app; a web browser interface and a visual display on a communal screen. The user interface 70 can be arranged to give instructions and other alerts to users to take actions, such as to open a window or door, to reduce the number of people in a room, or the like. The workflow modifiers and energy efficiency modifiers are also used by the workflow engine 16 to select the instructions and other alerts sent to the user interface 70. For example, if the external environmental information 40 indicates that it is much warmer outside than is desired for indoor conditions and the pollen count is high, the workflow engine 16 may apply a workflow modifier and energy efficiency modifier that selects automated workflow to increase the filtration of recycled air through the HVAC system via the workflow interface 50, rather than an instruction through the user interface 70 to open a window. Alternatively, the energy efficiency modifiers can be configured based on energy cost. If energy costs rise, a customer can strengthen the effect of energy efficiency modifiers (either creating new modifiers or changing the action of existing modifiers) in order to prioritise interventions that keep the space healthy but which maximise energy efficiency. For example, if energy costs rise 20%, an energy efficiency modifier might be used which prioritises increased circulation of recycled air via the HVAC system when outdoor temperatures are cold or hot compared to desired indoor temperature, rather than to increase the percentage of fresh air being ventilated through the HVAC system. This enables interventions to be taken to keep the space healthy, whilst increasing the energy efficiency.

The rules engine 14 of the processor unit 10 operates on the sensor signals that it receives from the environmental sensors 30, from the external environmental information 40, from the standards data within the standards library 21, the data within the infrastructure library 22, and the modifiers in the modifiers library 26 contained within the database 20, to assess whether there is an elevated risk to the enclosed space, and to generate a technical healthiness rating for the enclosed space or for a part of that enclosed space. If there is an elevated risk, the rules engine 14 will assess that risk with respect to any intervention which might be made. In this embodiment there are three elevated risk levels: 1. “elevated risk—no action required”, in which a risk factor is elevated, but where it is not yet at a level where intervention is required to address it; 2. “elevated risk—action required”, in which an intervention is required in order to reduce the elevated risk, although the risk is within the level of standards set out in the standards library 21; and 3. “elevated risk—unhealthy” in which an intervention is required in order to reduce the elevated risk, and the level of risk exceeds the accepted limits set out in the standard stored within the standards library 21.

If the elevated risk is in either of the risk levels requiring action, the processor unit 10 will assess the available interventions from the infrastructure library 22 of the database 20, in combination with the sensor signals from the environmental sensors 30, taking account of the workflow modifiers and energy efficiency modifiers, if any, and the rating modifiers, if any, and will determine the most appropriate type and strength of intervention or interventions. It will then send details of the intervention or interventions from a processor output to a workflow interface 50 in the form of a workflow instruction, and the workflow interface 50 will effect the intervention by applying appropriate workflows, for example, to the building management system 60, to make the necessary intervention. The workflow interface 50 will also wait for a verification signal 25 from the building management system 60, and pass this verification signal back to the processor unit 10 by way of handshake. While, for most interventions, these steps will be entirely automatic, there is the possibility of human interaction with the process. For example, if the intervention is to manually open windows, or to reapply a surface protection treatment, manual interaction may be required, for example, to instruct a building manager or specialist to carry out those interventions, and once done, to send a verification that the intervention has been completed.

The processor unit 10 is also able to validate the intervention by assessing the effect of the intervention through monitoring the sensor signals from the environmental sensors 30. If the elevated risk reduces, it will eventually be able to send a signal to the workflow interface 50 withdrawing the intervention, and the workflow interface 50 may operate with the building management system 60 to withdraw that intervention. This may be used to increase energy efficiency. If, on the other hand, the elevated risk continues to increase, or is not reduced, the processor unit 10 will select an alternative or an additional intervention to be enacted by the workflow interface 50 in order to continue to seek to reduce the elevated risk.

Whether or not the elevated risk reduces, the effects of the intervention together with all of the factors which led to the processor unit 10 choosing a particular intervention are recorded in the workflow response library 25 of the database. This data can be analysed by the processor unit and the rules within the rules engine 14 can be modified in order to improve the rules and modifiers for future use. Furthermore, machine learning and AI technology can be used in order to assist with the improvement of the rules and modifiers over time (including automatically editing the rules within the rules engine 16 and modifiers with the library of modifiers 26) so that interventions and workflow become continuously more effective. This enables continuous improvement of the risks to health and wellbeing in the enclosed space.

So far, the interventions described above are reactive to the detection of elevated risks. However, one of the benefits of recording interventions in the workflow response library 25 is that the processor unit can operate to predict when elevated risks will occur and make interventions as a proactive measure to mitigate those predicted elevated risks which have not yet manifested. Machine learning and AI can be used to analyse patterns in all of the data stored in the database 20 in order to determine how elevated risks may be predicted, and then to automatically edit the rules engine 14 in order to generate interventions and workflow that prevents likely elevated risks before they have arisen. Data collected from a growing number of premises over time in the database 20 may be used as training data for machine and learning and AI. This learning can be used to deploy improved reactive and proactive workflow more rapidly to a wider range of premises over time. For example, data stored in the database 20 may include data on the changing levels of carbon dioxide and occupancy of spaces, the volume of those spaces, and settings on HVAC systems controlled through the workflow interface 50 and deployed in those spaces. This data can be used to estimate air changes per hour and to learn how to better control the HVAC system as occupancy in the space varies. This enables machine learning to direct the workflow engine to deliver proactive workflow before elevated carbon dioxide levels arise when the space has increased occupancy.

The intervention that is determined by the processor unit 10 might be a manually applied intervention, in which case the workflow interface will notify a person of the need to carry out the determined intervention. Alternatively, the person may be notified of the need to carry out the intervention through the user interface 70.

The user interface has another purpose, which is to display the technical healthiness rating. The technical healthiness rating (THR) is an objective technical measure of the risk factors in the enclosed space, the elevation of which reflects the impact of the risk factors on the health of the occupants of the enclosed space. If the technical healthiness rating is displayed to the occupants of the enclosed space, they are able to see how healthy the enclosed space, or part of the enclosed space, is with reference to the risk factors, based on the sensor signals from the environmental sensors 30. The THR can be displayed as a numerical score and/or as a category. The THR may also be displayed either before rating modifiers are applied or after they are applied, the latter being used to show how well a building is performing relative to the external conditions outside. The purpose of the THR is to provide confidence to users that the enclosed space is free from significant environmental risks to their health. In one embodiment, the categories of Technical Healthiness Ratings for an enclosed space include: ‘Healthy’/Green; ‘Action Required’/Amber; and ‘Unhealthy’/Red. In this case, ‘Healthy’/Green can be displayed when the risk is not elevated, or when the risk is elevated to level 1 “elevated risk—no action required”. ‘Action Required’/Amber can be displayed when the risk is elevated to level 2 “elevated risk—action required”. ‘Unhealthy’/Red can be displayed when the risk is elevated to Level 3 “elevated risk—unhealthy”. Other embodiments can be envisaged with more or fewer categories, and with different expressions of the rating. The specific configuration for how the technical healthiness rating is presented for a particular space will depend upon the needs of the users and of management of that particular space.

The Technical Healthiness Ratings (both numerical and categories) are measured and reported in two ways:
‘Snapshot’—provides the Technical Healthiness Rating at a point in time, and is continually updated by the rules engine 14 to reflect the detection of current Elevated Risks at that moment in time;
‘Rolling Average’—provides a calculated average Technical Healthiness Rating over a defined period of time using the sensor data storage 24. The Rolling Average Technical Healthiness Rating is repeatedly re-calculated by the rules engine 14 and reported as time progresses. For example, the technical healthiness rating may be calculated as a daily, weekly or monthly rolling average.

The technical healthiness rating is calculated by collating inputs that are relevant to the risks, which include (but are not limited to) the sensor signals, external risk factors, risk modifiers and rating modifiers. The standards, the external risk factors, the risk modifiers and the rating modifiers are used to determine:
a) a level for each risk at or below which there is deemed to be no material
risk to human health and wellbeing, but above which action may be needed to ensure that a material risk to human health and wellbeing does not arise;
b) a level for each risk above which a more significant risk to human health and wellbeing may arise if individuals are exposed to those risks for a sustained period of time.

Each of the risks are then compared to the levels (a) and (b) above, and this is used to calculate a rating for each of them. The technical healthiness rating can be determined for each zone within a space or building (e.g. by using measurements provided by individual air monitors or surface tests in a particular room), or as an average for the space or premises as a whole (e.g. by taking an average for all monitors and surface tests for the premises).

One illustrative method of calculating the numerical score to rate each internal risk factor is as follows (either individually for each measurement recorded, or the average measurements for the premises as a whole):
where the level of an internal risk factor is at or below the level in (a) above, the rating is determined as 100%;
where the level of an internal risk factor is at or above the level in (b) above, the rating is determined as 0%;
where the level of an internal risk factor lies between the levels in (a) and (b), the rating is determined on a pro-rata basis (e.g. if the internal risk factor is measured as being half-way between level (a) and level (b), the rating would be calculated as 50%);

The ratings for the individual internal risk factors are then weighted and combined to calculate an overall technical healthiness rating for the space or premises as a whole. For example, this may be done by weighting the contribution of the individual percentages and then summing them in order to calculate an overall percentage. The weightings for the ratings of each internal risk factor may be modified by the external risk factors, the risk modifiers or the rating modifiers. For example, the presence of a pandemic with high levels of local infection rates may be used to increase the weighting for the internal risk factor related to people density and dwell time in confined spaces.

The levels specified in (a) and (b) above may also be used to determine the categories that are assigned to the overall THR calculated and presented to users and managers of the space. For example, one way this is done is:
A technical healthiness rating below the level in (a) above may be categorised as ‘Healthy’
A technical healthiness rating above the level in (a) but below the level in (b) above may be categorised as ‘Attention needed’
A technical healthiness rating above the level in (b) may be categorised as ‘Improvement needed’

Regardless of the precise way in which the technical healthiness rating is calculated and categorised, the identification of elevated risks for the internal risk factors being assessed and the consequent automatic generation of an intervention, together with machine learning and AI that makes continuous and predictive improvements, ensures that the resulting technical healthiness rating is continuously (and automatically) improved over time. This therefore continuously reduces risks to health and wellbeing in that space over time.

### Example 1

An example of the operation of this invention will now be described. In this example, the enclosed space is a public library which is equipped with a number of environmental sensors 30, including carbon dioxide sensors and people occupancy monitors for detecting people within the enclosed space, including their proximity, density, flow and dwell time. As the day passes, more people enter the library, and the present invention operates to monitor the enclosed space for risks to health, and to intervene as necessary. The process which is followed is illustrated in FIGS. 3 and 4.

The environmental sensors 30 monitor the risk factors within the library in step 301, 1A over a period of time, monitoring the carbon dioxide level and occupancy of the library. The sensor signals generated by the environmental sensors 30 are directed to the processor unit 10, together with several other inputs. The processor unit assesses 302 whether the sensor signals from the environmental sensors 30 represent an elevated risk 5B. This assessment 302 is carried out using: 1. standards 2A, 303, which are current Government standards and other available guidance for recommended carbon dioxide levels within a building, from the standards library 21. external environmental information 40 sourced externally 304 from the system via an Internet connection 41. At this point in time, the external environmental information 40 indicates that there is an influenza outbreak, and this causes a risk modifier to be applied 4B, 305, reducing the tolerance of the processor unit 10 for detection of elevated risks. The processor unit 10 carries out this assessment on an ongoing basis taking account of any changes to any of the inputs. We can assume that, at the beginning of the day, when the library is empty, or when it has a very low occupancy, the CO2 level will be low such that the processor 10 will not identify elevated risks. The processor unit 10 can carry out a calculation 7C, 306 of the Technical Healthiness Rating 307, and send that rating to the user interface 70 so that it can be displayed to people within the library as well as to people managing the library.

As the occupancy of the library increases, the CO2 level will increase, and the sensor signals output by the environmental sensors increases. Let us suppose that the CO2 level increases such that the processor unit 10 makes the assessment 5B that there is an elevated risk of viral transmission in the air owing to an increase in the density of people within the library, as indicated by the increase in the carbon dioxide level measured by the carbon dioxide sensor. The processor 10 can carry out a calculation 7C, 306 of the Technical Healthiness Rating 307, which will have increased, and send that increased rating to the user interface 70 so that it can be displayed to people managing the library and if required to people using the library. The processor unit 10 also carries out the step 10D, 310 of determining the most appropriate intervention to take to eliminate or reduce the elevated risk based on the interventions which are available to it from the infrastructure library 8A, 22. In this case, the infrastructure library 22 identifies that the 10 library has an HVAC system and an air purification system. It carries out this determination also taking into account workflow modifiers 311. In this instance, it is known from the external environmental information 40 that the external weather is warm with minimal external pollution levels and it is also known that the HVAC system of the library is more energy efficient 9D than its air purification system. Therefore, the processor unit will select the HVAC system as the most appropriate for intervention. In this situation, not only is the HVAC system switched on, but the processor unit chooses a setting of the HVAC system which is most appropriate to the elevated risk. The processor unit 10 generates an intervention signal which is passed 11D, 320 to the workflow interface 50, and it is the workflow interface which sends a signal to the building management system or HVAC system instructing it to switch on the HVAC system and to set it to a specific level. It will be appreciated that, were the external air to be highly polluted at this time, the air purification system would have been a more appropriate choice than the HVAC system, in spite of the difference in energy efficiency.

The workflow interface is arranged so as to receive a verification signal or handshake 13E, 321 from the building management system or HVAC system to confirm that the HVAC system has been turned on. That verification signal is returned to the processor unit 10 and appropriate action is taken if the verification signal is negative. This action might be to resend the intervention signal to the building management system or HVAC system, or to instruct the operation of the air purification system instead, or both.

The CO2 levels within the library should begin to drop, but the processor unit recalculates 14F, 325 the risk based on the sensor signals from the environmental sensors, and if the risk reduces to a level where it is no longer elevated, the Technical Healthiness Rating is improved, and the indicated rating on the user interface 70 is improved accordingly. If, however, the CO2 level does not drop, the processor unit 10 will identify this 327, and in 35 the first instance, the processor unit 10 can increase the level of intervention, either by increasing the mode of operation of the HVAC system, or by adding the air purification system, or both. In any event, the results of the recalculation 14F, 325 is stored 326. Furthermore, the processor unit 10 can use machine learning and AI, or notify the building managers to investigate the interventions which were not effective 16G, 330. For example, machine learning and AI may detect that at that particular time of day on that particular day of the week, there is a big increase in occupants caused by the presence of classes from the local college. The rules engine 14 can then be edited to generate workflow ahead of local college classes in order to reduce the risks of elevated risks arising in the future. Alternatively, this might indicate that there are problems with the HVAC system which requires maintenance.

The system may be able to learn from the effects of the interventions, and from when interventions are required. The processing unit 10 can review the data stored 326 in the workflow response library 25 to identify interventions which used to be effective, but are no longer effective, and to identify trends and patterns in the data and from the wider environment to identify and investigate the causes of interventions which do not have the expected outcome. As mentioned above, this might be an indication that the system being used for the interventions is no longer working properly, that the library needs to be updated with new information about the enclosed space, or the rules engine needs to be updated to optimise the rules. This learning can be carried out by machine learning and/or artificial intelligence 330 within the processor unit 10. Action can then be taken to capture and apply the learning 335, in this case by recording an elevated risk every Tuesday lunchtime during term time to pre-empt increased occupancy and to generate interventions ahead of the risk arising, or to cause the HVAC system to be serviced.

### Example 2

Another example of the operation of this invention will now be described as shown in FIG. 5. In this example, the enclosed space is a kitchen in which the surfaces are periodically treated with surface protection. The environmental sensors 30 may include surface swabs which are analysed by a handheld electronic device which assesses the level of organic compounds on the surfaces being tested. At present, a surface application services supplier would be automatically notified by the workflow interface 50 to periodically carry out a tour of the kitchen to test specified surfaces in turn, such as the door, the tap, the worktop surface, and the fridge door handle. After each swab/test, that test is analysed by the handheld unit, and the sensor signals from it are sent to the processor unit 10. The processor unit assesses whether the sensor signals from the environmental sensor 30 represent an elevated risk 5B. This assessment is carried out using: 1. standards from the standards library 21; and 2. external environmental information sourced externally from the system via an Internet connection 41. At this point in time, the external environmental information 40 indicates that there are no risk modifiers at the present time. The processor unit 10 carries out this assessment on an ongoing basis taking account of any changes to any of the inputs. The processor unit 10 carries out a calculation 7C, of the Technical Healthiness Rating, and sends that rating to the user interface 70 so that it can be displayed to people within the kitchen as well as to people managing the kitchen.

At a point in time in this example, the level of organic compounds on the kitchen door is detected to be high when compared with the relevant standards 2A. The processor unit 10 makes the assessment 5B that there is an elevated risk of viral transmission from contact with the kitchen door, as indicated by the increase in the detected organic compounds measured by the sensor. The processor 10 can carry out a calculation 7C, of the Technical Healthiness Rating, which will have increased, and send that increased rating to the user interface 70 so that it can be displayed to people within the kitchen as well as to people managing the kitchen. The processor unit 10 also carries out the step 10D, of determining the most appropriate intervention to take based on the interventions which are available to it from the infrastructure library 8A, 22 and the factors that influence which intervention to select based on the workflow modifiers determined in 10D. In this case, the workflow response library 25 identifies that the kitchen door panel has been treated with surface protection 5 months ago, and in step 10D the processor unit 10 determines that amongst the available interventions are to re-treat the surface of the door panel or to apply an adhesive anti-viral pad. The processor unit 10 identifies in step 10D that the retreatment of surface protection is the most cost-effective option. The processor unit 10 selects retreatment of surface protection as the preferred intervention and generates an intervention signal which is passed 11D to the workflow interface 50, and it is the workflow interface which sends an automatic notification to the surface application services supplier instructing it to re-apply the surface protection to the kitchen door.

The workflow interface is arranged so as to request a verification notification, either by email or via the workflow interface 50 or handshake 13E from the surface application services supplier to confirm that the surface protection has been reapplied. That verification signal is returned to the processor unit 10 and appropriate action is taken if the verification signal is negative. This action might be to resend the intervention signal to the surface application services supplier and the people responsible for managing the kitchen.

Once the surface protection has been reapplied, surface testing will be applied and repeated periodically. The processor unit recalculates 14F the risk based on the sensor signals from the surface testing, and if the risk reduces to a level where it is no longer elevated, the Technical Healthiness Rating is improved. Furthermore, the processor unit 10 can automatically notify the building managers if the intervention was not effective, so that they can investigate the reasons.

The system may be able to learn from the effects of the interventions, and from when interventions are required. The processor unit 10 can review the data stored in the workflow response library 25 to identify interventions which used to be effective, but are no longer effective, and to identify trends and patterns in the data and from the wider environment to identify and investigate the causes of interventions which do not have the expected outcome. This might be an indication that the surface protection is not as durable as expected so that it can be investigated. In this case, mechanical wear on the door is higher than expected, so the frequency with which surface protection is applied is increased. The infrastructure library then needs to be updated with new information about the enclosed space and the required frequency of surface treatment for particular locations, or the rules engine needs to be updated to optimise the rules. In addition, this may mean that in the long-run it is more cost effective to apply an adhesive anti-viral pad, rather than to keep re-treating the door panel on a more regular basis. This assessment and learning can be carried out by machine learning and/or artificial intelligence within the processor unit 10, which will have access to information on unit costs of retreating the surface protection and unit costs of supplying and applying anti-viral pads within the infrastructure library 22.

### Example 3

Another example of the operation of this invention will now be described. In this example, the enclosed space is an office having more than one meeting room. The meeting rooms are equipped with environmental sensors 30 including carbon dioxide sensors for detecting the carbon dioxide level in the air, and people occupancy monitors (such as from video management systems) which monitor the number of occupants within the meeting rooms.

The office also includes a meeting room booking system by which meeting rooms are reserved for meetings. The present invention operates to monitor the meeting rooms for health risks, and to intervene as necessary. The process which is followed is illustrated in FIGS. 6 and 7.

The environmental sensors 30 monitor the risk factors 1A within the meeting rooms in step 701 shown in FIG. 7. This monitoring takes place over a period of time, monitoring the carbon dioxide level in each meeting room, and measuring the occupancy of each meeting room. The sensor signals generated by the environmental sensors 30 are directed to the processor unit 10, together with several other inputs. The processor unit assesses in step 702 whether the sensor signals from the environmental sensors 30 represent an elevated risk 5B. This assessment 702 is carried out using: 1. standards 2A, 703, which are current Government standards for recommended carbon dioxide levels in an office, from the standards library 21; and 2. external environmental information 3A sourced 10 externally 704 from the system via an Internet connection 41. At this point in time, the external environmental information 40 indicates that there is a Covid-19 pandemic, and this causes a risk modifier to be applied 4B, 705, reducing the tolerance of the processor unit 10 for detection of elevated risks. The processor unit 10 carries out this assessment on an ongoing basis taking account of any changes to any of the inputs. We can assume that, at the beginning of the day, before any meetings have taken place, or when the occupancy of the meeting rooms is very low, the carbon dioxide level will be low, such that the processor 10 will not identify any elevated risks in the meeting rooms. The processor unit 10 continuously carries out a calculation 7C, 706 of the technical healthiness rating 707 of a meeting room, and sends that rating to the user interface 70 so that it can be displayed to people within the meeting room, as well as to people managing the meeting rooms.

At the beginning of a meeting in a first meeting room, the occupancy of that meeting room increases, which will be detected by the occupancy monitor, and the carbon dioxide level will increase, which will be detected by the carbon dioxide sensor. The environmental sensors 30 generate sensor signals which increased to reflect this. Let us suppose that the carbon dioxide level increases such that the processor unit 10 makes the assessment 5B that there is an elevated risk of viral transmission in the air owing to an increase in the number of people in the meeting room, as indicated by the increase in the carbon dioxide level measured by the carbon dioxide sensor. The processor unit 10 can carry out a calculation 7C, 706 of the technical healthiness rating, 707, which will have deteriorated, and send that increased rating to the user interface 70 so that it can be displayed to people within the first meeting room as well is to people managing the meeting rooms. The processor unit 10 also carries out the step 10D, 710 of determining the most appropriate intervention to take based on the interventions which are available to it from the infrastructure library 8A, 22, 713. In this case the infrastructure library 22 identifies that the meeting rooms have an air ventilation system, that it has a meeting room booking system which holds information on room bookings and the number of people booked to attend each meeting, and that the meeting room booking system stipulates 15 minute gaps between meetings with the door to the meeting room kept open to reduce the carbon dioxide levels between meetings. In this example, the interventions have not been successful in removing the elevated risks, and an elevated risk remains at the end of the meeting. The determination of the most appropriate intervention in this situation also takes into account workflow modifiers 711. In this instance, it is known from the infrastructure library 22 that the air ventilation system is already set to maximum and cannot be increased further, and that the door to the meeting room is already open because these were the interventions that were in place at the end of the meeting. Thus, there are no further workflow options available. In this example, there are also no input energy efficiency modifiers identified 9D. On the basis of this, the processor unit 10 will select the most appropriate remaining intervention, which is to move the subsequent meeting to a different meeting room 11D in order to give more time for the carbon dioxide levels in the first room to reduce than the 15 minutes that is normally stipulated. This 15 request is passed 720 to the workflow interface 50, and it is the workflow interface which sends a signal to the meeting room booking system requesting it to switch the subsequent meeting to another meeting room in order to leave the first meeting room vacant beyond the stipulated 15 minutes. In this embodiment, the meeting room booking system sends a verification signal 12A, 714 to the workflow interface to enable a digital handshake 20 13E,721 to verify the workflow has taken place.

The carbon dioxide levels within the first meeting room should begin to drop after the meeting is concluded, but the processor unit continues to recalculate 14F, 725 the risk based on the sensor signals from the environmental sensors. The time that it takes for the elevated risk to no longer be measured in step 14F, 725 is stored 726 in the workflow response library 25, along with the carbon dioxide measurements and occupancy of the first meeting room.

The system can then learn from previous situations. The processor unit 10 can review the data stored in the workflow response library 25 to identify 15F, 727 repeat instances where elevated risk is detected in the meeting room which cannot be resolved through increasing ventilation. This review can apply machine learning/AI 16G 730 to identify situations where this occurs and its correlation with the number of people attending a meeting. If, for example, it identifies that the ventilation system, even when operating a maximum level, is not sufficient to avoid the risk level from becoming elevated when there are 5 people or more in the meeting room for 1 hour or more, even when the door is kept open during the meeting. In addition, if it is found that the CO2 levels do not subside after such a meeting within 15 minutes of it finishing. The processor unit instructs 735 the meeting room booking system to change its rules to increase the vacancy gap following meetings with 5 people or more from 15 minutes to 30 minutes. Further monitoring and workflow following this rule change will then assess whether 30 minutes is sufficient, or whether any further rule changes in the meeting room booking system are required.

### Example 4

An example of the operation of this invention will now be described. In this example, the enclosed space is an office building which is equipped with a number of environmental sensors 30, including humidity sensors, PM2.5 sensors and people occupancy monitors for detecting people within the enclosed space, including their location within the building. There is also an external air monitor on the roof of the building which measures and provides external environmental information 40 about the outdoor air entering the inlet of the building's HVAC system, including its temperature, humidity and levels of PM2.5. As the day passes, more people enter the office building, and the present invention operates to monitor the enclosed space for risks to health, and to intervene as necessary. The process which is followed is illustrated in FIG. 8.

The environmental sensors 30 monitor the risk factors within the building in step 801 over a period of time, monitoring the humidity, PM2.5 and occupancy across the different spaces within the building. The sensor signals generated by the environmental sensors 30 are directed to the processor unit 10, together with several other inputs. The processor unit assesses 802 whether the sensor signals from the environmental sensors 30 indicate an elevated risk. This assessment 802 is carried out using standards 803, which are current Government standards and other available guidance for recommended humidity and PM2.5 levels within a building, from the standards library 21. The processor unit 10 carries out this assessment on an ongoing basis taking account of any changes to any of the inputs. The processor unit 10 can carry out a calculation 806 of the Technical Healthiness Rating 807, and send that rating to the user interface 70 so that it can be displayed to people within the offices as well as to people managing the building.

Let us consider Meeting Room 1 within the office building. Let us suppose that, whilst Meeting Room 1 is occupied as detected by the occupancy monitors, the humidity level in Meeting Room 1 falls such that the processor unit 10 makes the assessment 802 that there is an elevated risk of viral transmission in the air owing to overly dry indoor air. Let us also suppose that, at the same time, the level of PM2.5 in Meeting Room 1 rises such that the processor unit 10 makes the assessment 802 that there is an elevated risk to health due raised levels of PM2.5. The processor unit 10 can carry out a calculation 806 of the Technical Healthiness Rating 807, which will have deteriorated, and send that deteriorated rating to the user interface 70 so that it can be displayed to people managing the building and if required to people using Meeting Room 1, for example via an in-room display. The processor unit 10 also carries out the step 810 of determining the most appropriate intervention to take to eliminate or reduce the elevated risk based on the interventions which are available to it from the infrastructure library 22. In this case, the infrastructure library 22 identifies 811 that the building has an HVAC system and an IoT-enabled air purification device in Meeting Room 1, both of which can be controlled remotely by the workflow engine 16. It carries out this determination also taking into account workflow modifiers 812. In this instance, it is known from the external environmental information 40, 813 that the temperature of the external air is 0° C. whereas the desired temperature indoors is 20° C. The workflow engine 16 searches the library of modifiers 26 and identifies an energy efficiency modifier 814 that deprioritises and restricts an intervention to increase fresh air ventilation through the HVAC system where the outdoor temperature is more than 15° C. below the desired indoor temperature, in order to conserve energy usage. It is also known from external environmental information 40, 813 that the humidity of the outdoor air is particularly dry and contains raised levels of PM2.5. The infrastructure library 22 shows that there is no ability for the building's HVAC system to increase the humidity of external air entering the building. The workflow engine 16 also identifies further workflow modifiers 812 that deprioritise and restrict interventions to increase fresh air ventilation through the HVAC system where outdoor air is overly dry or has raised levels of PM2.5, both of which are the case in this example. As a result of the energy efficiency modifier 814 and other workflow modifiers 812, the workflow engine 16 selects an alternative intervention which is to send a signal to the workflow interface 50 to turn the air purification device in Meeting Room 1 up to a ‘high’ setting 815. Once turned up, the air purification device removes bacteria, viruses and PM2.5 in the air in Meeting Room 1, so although low humidity may persist, the risk of illness transmission from dry air is reduced by the intervention. The rules engine 14 searches the library of modifiers 26 to identify a risk modifier 816 that relaxes the thresholds for detecting elevated risks in Meeting Room 1 in relation to illness transmission in the air, due to the fact that the air purification device is turned up to ‘high’. The rules engine 14 also identifies a rating modifier 816 that improves the THR in Meeting Room 1 to reflect the current mode of the air purification device. The air purification device sends a signal via the internet to the workflow interface so that the workflow engine 16 is able to verify that the intervention has been carried out 817. The rules engine 14 continues to calculate elevated risks and the THR in Meeting Room 1, and in this case the intervention using the air purification device successfully eliminates the elevated risks in Meeting Room 1 by reducing PM2.5 and modifying the risk level arising due to low humidity. The rules engine 14 is thus able to validate 818 that the workflow has achieved the desired outcome.

Over time, various similar scenarios arise, and the database 20 stores historical data on internal risk factors, external environmental information, risk modifiers, rating modifiers, energy efficiency modifiers, workflow modifiers, workflow verified and validated and workflow which is not verified nor validated. Machine learning and AI is then applied to the data to identify improvements. As an example, AI identifies 819 from the historical data that when occupancy in Meeting Room 1 exceeds 10 persons for more than one hour, the carbon dioxide levels in Meeting Room 1 climb to such levels that the air purification device is no longer sufficient to avoid an elevated risk being detected and a sub-optimal THR being reported. AI then seeks to increase fresh air ventilation through the HVAC system in small increments to test the impact 820 for Meeting Room 1. In this example, AI identifies that increasing fresh air ventilation by 10% is sufficient to remove the elevated risk in Meeting Room 1 and return the THR to healthy. AI deploys this learning by deploying a workflow modifier 821 that increases fresh air ventilation by 10% when occupancy in Meeting Room 1 is 10 or more for 50 minutes or longer. In this way, AI has been able to use historical data to update the workflow modifiers such that an elevated risk or suboptimal THR in Meeting Room 1 is proactively prevented from similar conditions arising in the future.

### Example 5

An example of the operation of this invention will now be described. In this example, the enclosed space is a canteen within a large industrial facility. The canteen is equipped with environmental sensors 30, including occupancy heat-mapping monitors showing capacity usage across the canteen, and scheduled completion of surface hygiene tests. Over time, surface hygiene tests are carried out to monitor the canteen for risks to health, and to intervene as necessary. The process which is followed is illustrated in FIG. 9.

The environmental sensors 30 monitor the risk factors within the building in step 901 over a period of time, monitoring the levels of surface hygiene in different locations at different times, and occupancy levels in different parts of the canteen. The data from the environmental sensors 30 is directed to the processor unit 10. The processor unit assesses 902 whether the sensor signals from the environmental sensors 30 represent an elevated risk. This assessment 902 is carried out using standards 903, which are current Government standards and other available guidance for recommended surface hygiene levels to prevent the spread of illness, from the standards library 21. The processor unit 10 carries out this assessment each time surface hygiene tests are carried out. The processor unit 10 can carry out a calculation 904 of the Technical Healthiness Rating 905, and send that rating to the user interface 70 so that it can be displayed to people managing the canteen.

Let us suppose that the on the day of surface testing, the tables in row G of the canteen all show poor levels of surface hygiene such that the processor unit 10 makes the assessment 902 that there is an elevated risk of transmission of illness from these surfaces. The processor unit 10 can carry out a calculation 904 of the Technical Healthiness Rating 905, which will have deteriorated, and send that deteriorated rating to the user interface 70 so that it can be displayed to people managing the canteen. The processor unit 10 also carries out the step 906 of determining the intervention to take to eliminate or reduce the elevated risk based on the interventions which are available to it from the infrastructure library 22. In this case, the infrastructure library 22 identifies that the canteen is covered by an online cleaning schedule booking system where job requests can be posted by the workflow interface 50 to carry out enhanced cleaning. The workflow engine 16 selects an intervention to send a signal to the workflow interface 50, 907 to post a job request to the cleaning schedule booking system to carry out enhanced conventional cleaning of row G on a weekly basis. The rules engine 14 continues to calculate elevated risks and the THR in the canteen, including each time new surface hygiene testing is carried out. In this case, the intervention has not been successful. Although surface hygiene for row G is shown to be generally better the next time surface testing is carried out, there are still instance of elevated risks in row G, and in addition certain other locations in the canteen now have elevated risks. The rules engine 14 is thus not able to validate 908 that the workflow has fully achieved the desired outcome.

Machine learning and AI is then applied to the data to identify unvalidated outcomes 909 and to investigate improvements. In this example, AI identifies 910 from the historical occupancy data that when areas of the canteen have been occupied at greater than 80% capacity for more than 30 minutes, there is a high degree of correlation with elevated risks arising due to poor surface hygiene. AI creates a workflow modifier 912 that creates a new workflow option to request intensive cleaning of areas of the canteen where occupancy has exceeded 80% of capacity for more than 30 minutes. In this way, AI has been able to use historical data to update the workflow modifiers such that an elevated risk or suboptimal THR in the canteen is rapidly mitigated and the efficiency of cleaning schedules is optimised using occupancy data.

The processor unit 10 may be a personal computer, a laptop, a server housed at the premises of the installation of the system, a server located remotely and accessed via the Internet, a general purpose computing device or by some other appropriate computing device. The present application is not limited to a particular type of processor unit. The processor unit 10 will include one or more computer processors, memory, and interfaces which allow access to other resources, such as the database 20, the Internet 41, the environmental sensors 30, user interfaces 70, and the workflow interface 50. Some of these interfaces will be bidirectional. The processor unit 10 is described as having 3 functional engines, and these engines can be run on a single computer processor, or on multiple computer processors, as appropriate. The computer processor might, for example, be one or more microprocessors, microcontrollers, multicore processors, graphical processors, ASICs or the like. The computer processor communicates with the memory, and with the other components via the interfaces in order to carry out the invention disclosed in this patent application.

1. A system for mitigating risk in an enclosed space comprising:
a processor unit having a rules engine and a workflow engine;
an infrastructure library accessible by the processor unit and holding a plurality of available interventions which can be enacted by the system to mitigate risk;
a plurality of environmental sensors, each arranged to supply the processor unit with sensor signals indicating a sensed risk factor of the enclosed space; and
a workflow interface arranged to instruct the operation of an intervention;
wherein the rules engine is configured to process the sensor signals from the plurality of sensors against a set of rules to determine if the risk is elevated to an elevated risk level where mitigating action is required;
wherein the workflow engine is configured to select one or more of the available interventions from the infrastructure library when the risk is elevated to the elevated risk level where mitigating action is required; and
wherein the processor unit is configured to generate a workflow instruction signal when an intervention has been selected and to send that workflow instruction signal to the workflow interface.

2. A system according to claim 1, wherein the rules engine is arranged to generate a technical healthiness rating based on its determination of risk, the technical healthiness rating being an objective technical measure of the risk factors in the enclosed space.

3. A system according to claim 1, further comprising an indicator for generating an indication of the technical healthiness rating.

4. A system according to claim 1, further comprising a standards library which holds a set of pre-programmed standards for the enclosed space to meet.

5. A system according to claim 1, further comprising a library of modifiers including at least one of: risk modifiers;
rating modifiers, workflow modifiers; and energy efficiency modifiers.

6. A system according to claim 5, wherein the rules engine is configured to modify the elevated risk level of the set of rules based on the risk modifier in order to modify the risk level at which mitigation action is required.

7. A system according to claim 5, wherein the workflow engine is configured to modify the intervention based on the workflow modifier and/or the energy efficiency modifier in order to modify the intervention that is required.

8. A system according to claim 1, further comprising means for sourcing external environmental information and supplying that information to the processor unit.

9. A system according to claim 1, further comprising a sensor data storage for storing sensor data corresponding to the sensor signals from the sensors over a period of time.

10. A system according to claim 1, wherein the workflow interface is arranged to source verification that an intervention has been implemented.

11. A system according to claim 1, wherein the processor unit is configured to analyse the effect of the intervention on the sensed risk factors of the enclosed space from the environmental sensors, and if further intervention is required, to select a further intervention.

12. A system according to claim 1, further comprising a workflow response library which stores historic interventions which have taken place, together with the (i) verification and/or (ii) validation of those interventions.

13. A system according to claim 12, wherein the processor is further configured to analyse the effect of an intervention on the sensed risk factors of the space from the environmental sensors, and to record the result in the workflow response library; and wherein the processor unit is also configured to use the contents of the workflow response library when selecting one or more mitigation actions.

14. A system according to claim 12, wherein the processor unit includes a machine learning or AI engine arranged to apply machine learning or AI to the analysis of the effect of an intervention on the sensed risk factors of the space.

15. A system according to claim 13, wherein the processor unit or machine learning or AI engine are arranged to edit the set of rules in the rules engine and/or the workflow engine to change their responses.

16. A system according to claim 15, wherein the editing of the set of rules in the rules engine and/or the workflow engine changes the risk modifiers, workflow modifiers, rating modifiers and/or energy efficiency modifiers.

17. A system according to claims 14, wherein the machine learning or AI engine is arranged to analyse the sensor data stored in the sensor data storage and to identify repeat instances where elevated risk is detected so as to cause the system to pre-empt the elevation of risks with the implementation of an intervention.

18. A system according to claim 1, wherein the workflow interface is connected to a plurality of different systems for carrying out different interventions.

19. A method of mitigating risk in an enclosed space comprising:
processing, using a rules engine, the sensor signals from a plurality of environmental sensors, each sensor arranged to supply the rules engine with sensor signals indicating a sensed risk factor of the enclosed space against a set of rules to determine if the risk is elevated to an elevated risk level where mitigating action is required;
selecting one or more available interventions which can be enacted by the system from an infrastructure library when the risk is elevated to the elevated risk level where intervention is required; and
generating a workflow instruction signal when an intervention has been selected and sending that workflow instruction signal to a workflow interface which is arranged to instruct the operation of the selected intervention.
